(19) United States
(12) Patent Application Publication
(10) Pub. No.: US 2024/0170150 A1
(43) Pub. Date: May 23, 2024

(54) MEDICAL INFORMATION PROCESSING APPARATUS AND METHOD

(71) Applicant: Canon Medical Systems Corporation, Otawara-shi (JP)

(72) Inventor: Yusuke KANO, Nasushiobara (JP)

(73) Assignee: Canon Medical Systems Corporation, Otawara-shi (JP)

(21) Appl. No.: 18/512,592

(22) Filed: Nov. 17, 2023

(30) Foreign Application Priority Data
Nov. 18, 2022 (JP) 2022-185156

(51) Int. Cl. G16H 50/20; G16H 10/60

(52) U.S. Cl. CPC G16H 50/20; G16H 10/60

(57) ABSTRACT

A medical information processing apparatus acquires multiple training samples. Each of the training samples includes a feature amount representing a condition of a subject, a type label of an event performed on the subject, and an effect label of the event. The apparatus acquires a knowledge base independent from the training samples. The processing circuitry assigns a knowledge label to at least one training sample among the training samples based on the knowledge base. The apparatus trains, based at least on the at least one training sample to which the knowledge label is assigned, a model that infers an effect of each type of an event. The at least one training sample to which the knowledge label is assigned includes the feature amount, the type label, the effect label, and the knowledge label.

### CROSS-REFERENCE TO RELATED APPLICATION(S)

This application is based upon and claims the benefit of priority from Japanese Patent Application No. 2022-185156, filed Nov. 18, 2022, the entire contents of which are incorporated herein by reference.

### FIELD

Embodiments described herein relate generally to a medical information processing apparatus and method.

### BACKGROUND

In individualized medical care, it is important to estimate therapeutic effects by correctly considering a causal relationship. To respond to this need, an attempt has been made to construct a causal inference model that estimates therapeutic effects of a medical event to be performed on a patient from a feature amount representing the condition of the patient. In regard to medical care, however, it may be difficult to collect large numbers of training samples for machine learning. Also, in order to improve the accuracy of the estimation of the therapeutic effects, it is desirable to utilize not only machine learning based on training samples but also existing medical knowledge acquired in the past.

### BRIEF DESCRIPTION OF THE DRAWINGS

FIG. 1 shows an example of a configuration of a medical information processing apparatus according to an embodiment.

FIG. 2 shows a procedure of medical information processing (a process of training a causal inference model).

FIG. 3 schematically shows the training process shown in FIG. 2.

FIG. 4 schematically shows an example of acquiring a knowledge base.

FIG. 5 shows a specific example of a training data set obtained after a knowledge label is assigned.

FIG. 6 schematically shows the entire training process performed on the causal inference model.

FIG. 7 schematically shows the details of a first loss function L_{Y} and a second loss function L_{K}.

FIG. 8 shows an example of a configuration of a medical information processing apparatus according to an application example 1.

FIG. 9 schematically shows a training process according to the application example 1.

FIG. 10 shows an example of a configuration of a medical information processing apparatus according to an application example 2.

FIG. 11 schematically shows a process of generating an artificial sample.

FIG. 12 schematically shows a process of determining whether or not to adopt the artificial sample.

FIG. 13 shows an example of a configuration of a medical information processing apparatus according to an application example 3.

FIG. 14 shows a procedure of an inference process performed by the medical information processing apparatus according to the application example 3.

FIG. 15 shows an example of a display screen of a therapeutic effect value and a recommended type according to the application example 3.

### DETAILED DESCRIPTION

A medical information processing apparatus according to an embodiment includes a first acquisition unit, a second acquisition unit, an assigning unit, and a training unit. The first acquisition unit acquires multiple training samples. Each of the multiple training samples includes a feature amount representing a condition of a subject, a type label of an event performed on the subject, and an effect label of the event. The second acquisition unit acquires a knowledge base independent from the multiple training samples. The assigning unit assigns a knowledge label to at least one training sample among the multiple training samples based on the knowledge base. The training unit trains, based at least on the at least one training sample to which the knowledge label is assigned, a model that infers an effect of each type of an event. The at least one training sample to which the knowledge label is assigned includes the feature amount, the type label, the effect label, and the knowledge label.

Hereinafter, a medical information processing apparatus, method, and program according to an embodiment will be described with reference to the accompanying drawings.

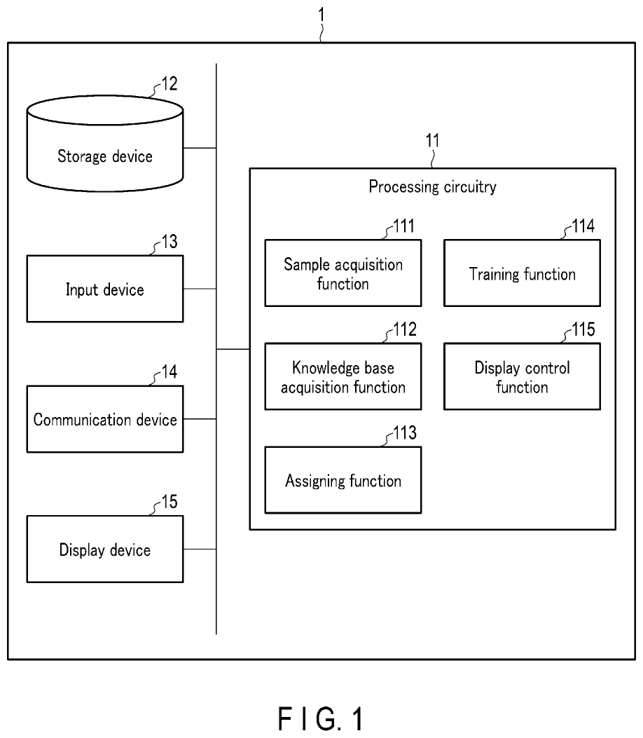

FIG. 1 shows an example of a configuration of a medical information processing apparatus 1 according to an embodiment. As shown in FIG. 1, the medical information processing apparatus 1 is an information processing terminal, such as a computer, that includes processing circuitry 11, a storage device 12, an input device 13, a communication device 14, and a display device 15. The processing circuitry 11, the storage device 12, the input device 13, the communication device 14, and the display device 15 are connected with one another via a bus so that they can input and output signals to and from one another.

The processing circuitry 11 includes processors such as a CPU (central processing unit) and a GPU (graphics processing unit). The processing circuitry 11 implements a sample acquisition function 111, a knowledge base acquisition function 112, an assigning function 113, a training function 114, a display control function 115, and the like by executing a medical information processing program. Note that the functions 111 to 115 may not be implemented by single processing circuitry. Multiple independent processors may be combined to form the processing circuitry so that the processors run programs to realize the functions 111 to 115, respectively. Besides, the functions 111 to 115 may be modularized programs that constitute a medical information processing program. These programs are stored in the storage device 12.

The storage device 12 is a ROM (read only memory), a RAM (random access memory), an HDD (hard disk drive), an SSD (solid state drive), an integrated circuit storage device, or the like that stores various kinds of information. Other than being one of the above-described storage devices, the storage device 12 may be a driver that reads and writes various kinds of information from and to, for example, a semiconductor memory device or a portable recording medium such as a CD (compact disc), a DVD (digital versatile disc), or a flash memory. The storage device 12 may be provided in an external computer connected via a network.

The input device 13 accepts various kinds of input operations from an operator, converts the accepted input operations to electric signals, and outputs the electric signals to the processing circuitry 11. Specifically, the input device 13 is connected to input devices such as a mouse, a keyboard, a trackball, a switch, a button, a joystick, a touch pad, and a touch-panel display. The input device 13 outputs, to the processing circuitry 11, electric signals corresponding to input operations to the input devices. A voice input device may be used as the input device 13. The input device 13 may also be an input device provided in an external computer connected via a network, etc.

The communication device 14 is an interface for transmitting and receiving various kinds of information to and from an external computer. The information communication performed by the communication device 14 follows standards appropriate for medical information communication such as DICOM (digital imaging and communications in medicine).

The display device 15 displays various kinds of information through the display control function 115 of the processing circuitry 11. For example, an LCD (liquid crystal display), a CRT (cathode ray tube) display, an OELD (organic electro luminescence display), a plasma display, or any other display can be suitably used as the display device 15. A projector may also be used as the display device 15.

The processing circuitry 11 acquires multiple training samples by implementing the sample acquisition function 111. Each of the multiple training samples includes a feature amount representing a condition of a subject, a type label of an event performed on the subject, and an effect label of the event. The subjects may be the same person or different people among the multiple training samples. The “subject” need not necessarily be an actual person, and may be an imaginary person obtained by statistical computing such as a statistically typical healthy person, a patient affected with a specified disease, a person with a specific age, a person with a specific gender, or a specific race.

The “feature amount” according to the embodiment is a numerical value, a sentence, a symbol, etc., representing the condition of the subject. The feature amount is information used as input data in machine learning. Typically, multiple types of feature amounts are included in a single training sample. The type of feature amount is herein referred to as a “feature amount type”. Specifically, the feature amount is a vector or a matrix that has multiple feature amount types combined with multiple numerical values (elements), etc., that respectively correspond to the multiple feature amount types. The number of elements of the feature amount according to the embodiment may be one.

The “event” according to the embodiment means a medical practice performed on the subject by medical staff, etc., and an action taken by the subject by him/herself. Various types of events are referred to as “event types”. The “type label” according to the embodiment is a numerical value, a character, a symbol, etc., representing the type of the event performed on the subject relating to the training sample, and means information used as correct data in machine learning. The “effect label” is a numerical value, a character, a symbol, etc., representing the therapeutic effect of the event performed on the subject relating to the training sample, and means information used as correct data in machine learning. The numerical value, character, symbol, etc., representing the therapeutic effect is referred to as a “therapeutic effect value”. The type of the therapeutic effect value may be a single type or multiple types. The type of the therapeutic effect value is referred to as a “therapeutic effect type”. The therapeutic effect type may be not only clinical outcomes such as a one-year survival rate, a six-month survival rate, major cardiovascular events (MACE: major adverse cardiac events), and cardiac function classification (NYHA: New York Heart Association classification), but also patient-reported outcomes, such as subjective symptoms and satisfaction with treatment, and economic outcomes, such as medical costs, medical resources, and a length of hospital stay.

By implementing the knowledge base acquisition function 112, the processing circuitry 11 acquires a knowledge base independent from the multiple training samples acquired by the sample acquisition function 111. The “knowledge base” according to the embodiment means a database in which existing medical knowledge is systematically collected. The knowledge base includes a type of recommended event, a degree of recommendation of the recommended event, and a feature amount representing the condition of a person to which the recommended event is applied. The term “independent” as used herein means that the knowledge base is not generated based on the training samples or that the training samples are not generated based on the knowledge base. The recommended event means an event recommended in the knowledge base.

By implementing the assigning function 113, the processing circuitry 11 assigns, based on the knowledge base acquired by the knowledge base acquisition function 112, a knowledge label to at least one training sample among the multiple training samples acquired by the sample acquisition function 111. The “knowledge label” according to the embodiment includes a type of recommended event and a degree of recommendation of the recommended event. Hereinafter, the type of recommended event is referred to as a “recommended type”. The knowledge label is used as correct data in machine learning.

By implementing the training function 114, the processing circuitry 11 trains, based at least on the at least one training sample to which the knowledge label is assigned, a causal inference model that infers an effect of each type of an event. The “at least one training sample to which the knowledge label is assigned” includes the feature amount, the type label, the effect label, and the knowledge label. The processing circuitry 11 may train a causal inference model based on a training sample to which no knowledge label is assigned, in addition to the “at least one training sample to which the knowledge label is assigned”. The “training sample to which no knowledge label is assigned” includes the feature amount, the type label, and the effect label.

By implementing the display control function 115, the processing circuitry 11 causes various kinds of information to be displayed on the display device 15. As an example, the processing circuitry 11 causes the result of training the causal inference model, the training samples, the knowledge base, the knowledge label, etc., to be displayed.

Hereinafter, an example of an operation of the medical information processing apparatus 1 according to the embodiment will be described.

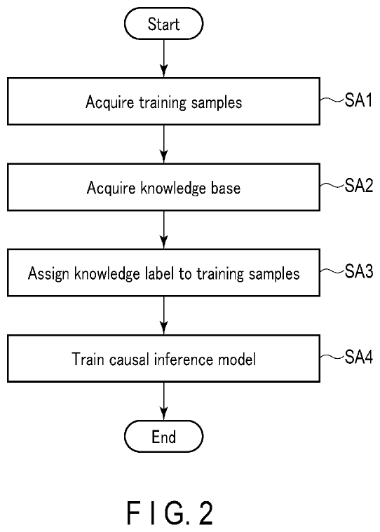

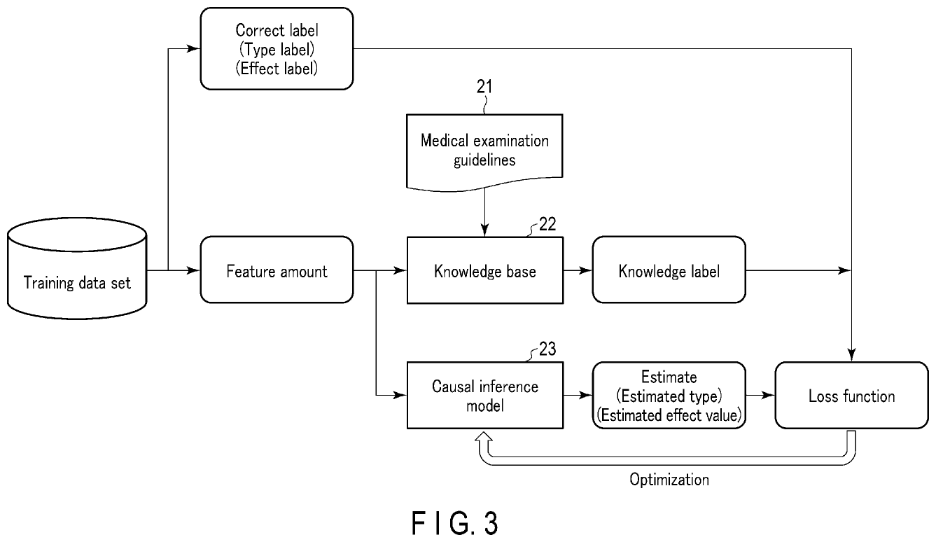

FIG. 2 shows a procedure of medical information processing performed by the processing circuitry 11 according to a medical information processing program. The medical information processing shown in FIG. 2 is assumed to be a process of training a causal inference model 23. FIG. 3 schematically shows the training process shown in FIG. 2.

As shown in FIG. 2, first, the processing circuitry 11 acquires multiple training samples by implementing the sample acquisition function 111 (step SA1). The multiple training samples each has a correct label and a feature amount. The correct label has a type label and a therapeutic effect value. A set of the multiple training samples is referred to as a “training data set”. The processing circuitry 11 acquires the training samples from a computer installed in a medical facility, etc., via the communication device 14. A verification sample that is used for verifying the result of the training may also be acquired separately.

After step SA1, the processing circuitry 11 acquires a knowledge base 22 by implementing the knowledge base acquisition function 112 (step SA2). Specifically, the processing circuitry 11 constructs the knowledge base 22 from medical examination guidelines 21 in step SA2. The medical examination guidelines 21 are sentence data of existing medical knowledge showing recommendations for typical feature amounts relating to target diseases. The recommendations are sentence items that present, for typical feature amounts of patients, medical practices (recommended events) that are appropriate or inappropriate for the patients. The degree of recommendation of the recommended events is associated with the recommendations. The processing circuitry 11 performs natural language processing, a statistical causation search, etc., on the medical examination guidelines 21 to assess the causal relationship between the recommended events and the feature amounts, and correlates the recommended events and the feature amounts that meet the causal relationship, and further correlates the degree of recommendation corresponding to the recommended events. As a result, the knowledge base 22 is constructed. The knowledge base 22 may be constructed using a different algorithm or manually. If the knowledge base 22 is already constructed, the processing circuitry 11 may import the knowledge base 22.

After step SA2, the processing circuitry 11 assigns a knowledge label to the training samples by implementing the assigning function 113 (step SA3). In step SA3, the processing circuitry 11 applies the feature amounts included in the training samples to the knowledge base 22 to specify recommended events corresponding to the feature amounts and the degree of recommendation, and assigns the training samples with the specified recommended events and the degree of recommendation as a knowledge label.

Herein, the acquisition of the knowledge base 22 (step SA2) and the assignment of the knowledge label (step SA3) will be explained by showing specific examples. The medical examination guidelines 21 according to the working example described below are assumed to be guidelines for valvular disease treatment, which targets a valvular disease.

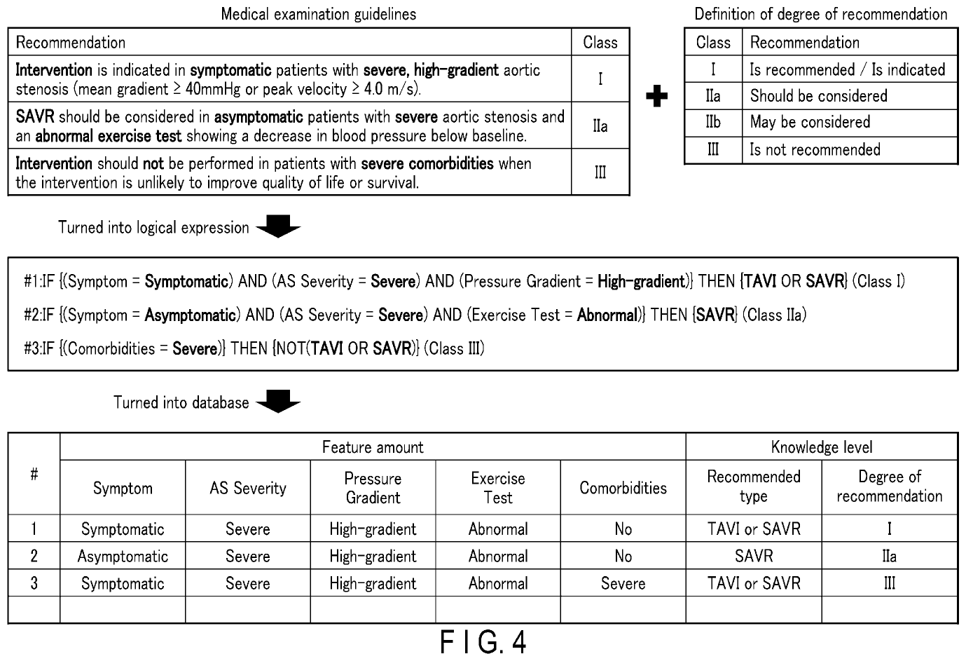

FIG. 4 schematically shows an example of acquiring the knowledge base 22. As shown in FIG. 4, the guidelines for valvular disease treatment have recommendation items and class items. The recommendation items express, using natural sentences, the relationship between the type of event recommended (recommended type) and the feature amount appropriate or inappropriate for the recommended type. The class items represent the degree of recommendation of the recommended type.

As an example, in the first case in the upper left figure of FIG. 4, the following is recorded as a recommendation item: “Intervention is indicated in symptomatic patients with severe, high-gradient aortic stenosis (mean gradient≥40 mmHg or peak velocity≥4.0 m/s). In this case, the recommended event is “intervention”, and the feature amounts appropriate for intervention are “symptomatic patients with high-gradient aortic stenosis (mean gradient≥40 mmHg or peak velocity≥4.0 m/s)”. The recommendation item of this case is associated with the degree of recommendation “I” as a class item.

As shown in the upper right figure of FIG. 4, the guidelines for valvular disease treatment define the degree of recommendation. The definition of the class “I” is “Is recommended” or “Is indicated”. The definition of the class “IIa” is “Should be considered”. The definition of the class “IIb” is “May be considered”. The definition of the class “III” is “Is not recommended”.

As shown in the middle figure of FIG. 4, the processing circuitry 11 performs natural language processing, a statistical causation search, etc., on the recommendation item and the class item of each case, and expresses, using a logical expression, the causal relationship between the feature amount and the recommended event of each case. As an example, the case #1 is expressed by a logical expression as in IF {(Symptom=Symptomatic) AND (AS Severity=Severe) AND (Pressure Gradient=High-gradient)} THEN {TAVI OR SAVR} (Class I). That is, the case #1 is converted into a logical expression that means “If the value of the feature amount type ‘Symptom’ is ‘Symptomatic’, the value of the feature amount type ‘AS Severity (aortic stenosis severity) is ‘Severe’, and the value of the feature amount type ‘Pressure Gradient’ is ‘High-gradient’, then, the recommended type is ‘TAVI’ or ‘SAVR’, which are interventions. “TAVI” is an abbreviation of transcatheter aortic valve implantation, and “SAVR” is an abbreviation of surgical aortic valve replacement. The logical expression relating to the guidelines for valvular disease treatment is an example of the knowledge base 22.

The processing circuitry 11 converts the logical expression representing the causal relationship between the feature amounts and the recommended events into a database (hereinafter referred to as a “guideline database”), as shown in the lower figure of FIG. 4. The guideline database has feature amount items and knowledge label items. The feature amount items have “symptom”, “aortic stenosis severity (AS severity), “pressure gradient”, “exercise test”, “comorbidities”, etc. The knowledge label items have the “recommended type” and the “degree of recommendation”, which are knowledge labels. The processing circuitry 11 converts the logical expression into a database by allocating the values of the feature amount items and the knowledge label items included in the logical expression to the guideline database. The guideline database relating to the guidelines for valvular disease treatment is an example of the knowledge base 22. In this manner, the knowledge base 22 relating to the guidelines for valvular disease treatment is constructed.

Once the knowledge base 22 is constructed, the processing circuitry 11 assigns a knowledge label to one or some of the training samples in the training data set. Specifically, the processing circuitry 11 compares the feature amounts of each training sample included in the training data set (herein referred to as “sample feature amounts”) with the feature amounts of each case included in the knowledge base 22 (herein referred to as “knowledge feature amounts”), and specifies a training sample that has sample feature amounts that match the knowledge feature amounts of each case. The processing circuitry 11 then adds the knowledge label of the case to the specified training sample. No knowledge label is assigned to a training sample that has sample feature amounts that do not match the knowledge feature amounts of all the cases included in the knowledge base 22. That is, a knowledge label is assigned only to one or some of the training samples included in the training data set. The process of assigning a knowledge label is thereby completed.

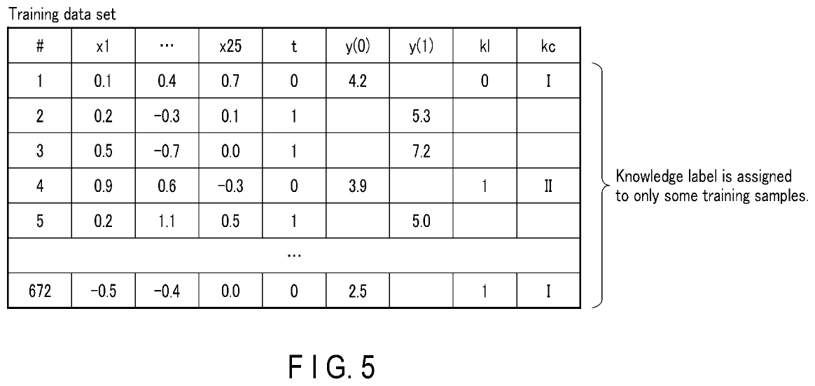

FIG. 5 shows a specific example of the training data set after assignment of a knowledge label. As shown in FIG. 5, the training data set relating to this specific example includes 672 training samples, training samples #1 to #672. The training data set includes items of feature amounts x1, x2, . . . , x25, type label t, therapeutic effect values y(0), y(1), and a knowledge label (recommended type kl, degree of recommendation kc). The type label t is assigned with “0” indicating that the type of event (medical practice) performed on a patient of the training sample is type 0 or assigned with “1” indicating that said type is type 1. If the type 0 is performed, a therapeutic effect value of the type 0 is allocated to the therapeutic effect value y(0), and if the type 1 is performed, a therapeutic effect value of the type 1 is allocated to the therapeutic effect value y(1). Since either one of the medical practice of the type 0 or the medical practice of the type 1 is performed on each patient, only either one of the therapeutic effect value y(0) or the therapeutic effect value y(1) will have a value. If the numerical values of the feature amounts x1, x2, . . . , x25 of each training sample match the numerical values of the feature amounts of the cases in the knowledge base 22, the numerical values of the recommended type kl and the degree of recommendation kc, which are the knowledge label of the cases, are allocated to the training samples.

As an example, the recommended type kl “0” and the degree of recommendation kc “I” are allocated to the training sample #1. The type label t of the training sample #1 is “0” and matches the recommended type kl “0”. Neither the recommended type kl nor the degree of recommendation kc is allocated to the training sample #2. While the recommended type kl “0” and the degree of recommendation kc “II” are allocated to the training sample #4, the type label t of the training sample #4 is “0” and does not match the recommended type kl “1”.

The training data set shown in FIG. 5 is merely an example, and the number of training samples, the number of elements of the feature amounts x (the number of feature amount types), the number of values that may be taken by the type label t, and the number of therapeutic effect values y (the number of therapeutic effect types) can be freely changed. The feature amount x and the therapeutic effect value y need not necessarily be represented by a numerical value, and may be represented by a character, a symbol, or the like.

After step SA3, the processing circuitry 11 trains the causal inference model 23 by implementing the training function 114 (step SA4). Simply put, the causal inference model 23 is a machine-trained model to which a feature amount x is input and from which an estimate of a therapeutic effect value (estimated effect value) y is output. In this case, the causal inference model 23 can be expressed simply by a mathematical formula, y=f(x). By implementing the training function 114, the processing circuitry 11 trains the causal inference model 23 using multi-task training including estimation of a knowledge label k and estimation of an estimated effect value y.

However, other than the estimated effect value y, the causal inference model 23 may output an -estimated type-t. The estimated type t means an estimated value of the type label of the event performed on a patient having a feature amount x. In the working example described below, the causal inference model 23 is assumed to be a machine-trained model to which a feature amount x is input and from which an estimated type t and an estimated effect value y are output. This causal inference model 23 can be expressed by two mathematical formulas, y=f(x) and t=g (x). In the process of training the causal inference model 23, the training parameters of the causal inference model 23 are optimized so as to reduce a loss assessed by a loss function L (y, t, k). The training parameters correspond to parameters such as a weight, a bias, and the like.

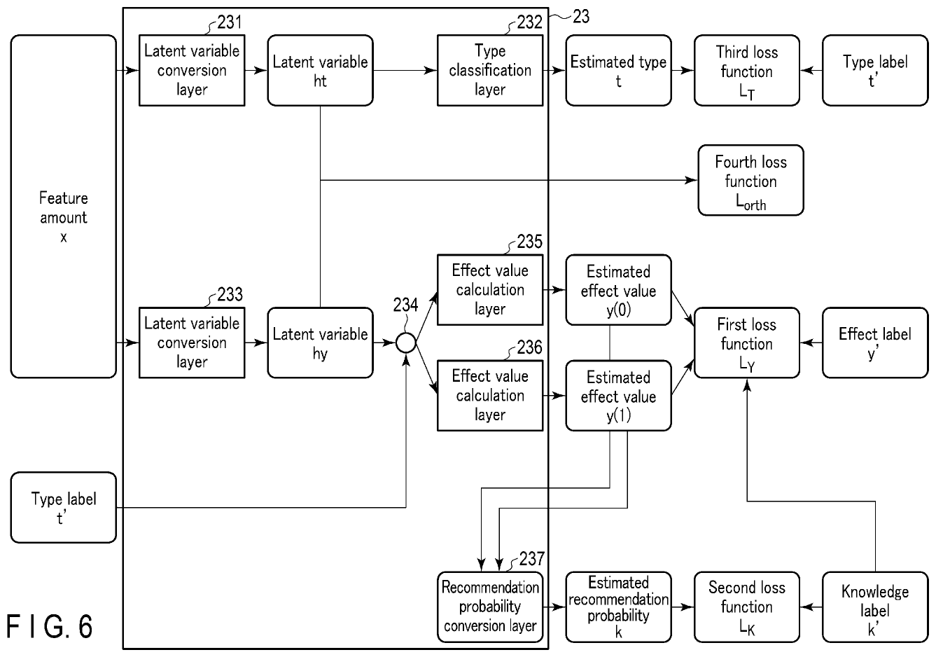

FIG. 6 schematically shows the entire training process performed on the causal inference model 23. As shown in FIG. 6, the training process has a first series that outputs an estimated type t and a second series that outputs estimated effect values y(0) and (y1).

Regarding the first series, the causal inference model 23 has a latent variable conversion layer 231 and a type classification layer 232. The latent variable conversion layer 231 is a network layer to which feature amounts x1 to x25 are input and from which a latent variable ht is output. The latent variable ht is a vector that has a dimension lower than the dimensions of the feature amounts x1 to x25. The network layer has one or more convolutional layers, totally coupled layers, pooling layers and/or other intermediate layers. The type classification layer 232 is a network layer to which the latent variable ht is input and from which an estimated type t is output. The estimated type t is a vector that has a combination of classification probabilities respectively corresponding to predetermined classified classes. The classified class means a class of the classification of classes in machine learning. The classified class relating to an estimated type corresponds to each type of event types. The classification probability of a predetermined event type is calculated as an estimated type t. “TAVI”, “SAVR”, or the like is set as an event type. The network layer has one or more convolutional layers, totally coupled layers, pooling layers and/or other intermediate layers.

Regarding the second series, the causal inference model 23 has a latent variable conversion layer 233, a distribution layer 234, effect value calculation layers 235 and 236, and a recommendation probability conversion layer 237. The latent variable conversion layer 233 is a network layer to which the feature amounts x1 to x25 are input and from which a latent variable hy is output. The latent variable hy is a vector that has a dimension lower than the dimensions of the feature amounts x1 to x25. The network layer has one or more convolutional layers, totally coupled layers, pooling layers and/or other intermediate layers.

The distribution layer 234 distributes the latent variable hy to the subsequent effect value calculation layer 235 and effect value calculation layer 236. The effect value calculation layer 235 is a network layer to which the latent variable hy is input and from which an estimate of a therapeutic effect of a type 0 (estimated effect value) y(0) is output. The effect value calculation layer 236 is a network layer to which the latent variable hy is input and from which an estimate of a therapeutic effect of a type 1 (estimated effect value) y(1) is output.

In the training process, the distribution layer 234 distributes the latent variable hy to both the effect value calculation layer 235 and the effect value calculation layer 236.

The recommendation probability conversion layer 237 is a network layer to which the estimated effect value y(0) and the estimated effect value y(1) are input and from which an estimated recommendation probability k is output. The estimated recommendation probability k is a vector of estimates of recommendation probabilities respectively corresponding to predetermined classified classes. The classified classes relating to estimated recommendation probabilities correspond to event types. The recommendation probability conversion layer 237 has a totally coupled layer and an activation layer that follows the totally coupled layer. The activation layer is a network layer that performs computing according to any activation function. In the case of performing two-class output of the estimated effect value y(0) and the estimated effect value y(1), the recommendation probability conversion layer 237 outputs the estimated recommendation probability k by applying a sigmoid function “Sigmoid” to a(y(0)−y(1))+b), as shown in the following formula (1) :

k=Sigmoid(a(y⁽⁰⁾−y⁽¹⁾)+b) (1)

As one example, in the case of performing multi-class output, the recommendation probability conversion layer 237 outputs the estimated recommendation probability k by applying a softmax function “Softmax” to a sum of a product of an estimated effect value matrix y and a weight matrix W and a bias b, as shown in the formula (2) below.

k=Softmax(Wy+b) (2)

The processing circuitry 11 trains the causal inference model 23 based on a training sample to which a knowledge label k′ is assigned and a training sample to which no knowledge label k′ is assigned. Specifically, the processing circuitry 11 calculates a loss function Lₜₒₜₐₗ based on the estimated type t, the type label t′, the latent variable ht, the latent variable hy, the estimated effect value y(0), the estimated effect value y(1), the effect label y′, the estimated recommendation probability k, and the knowledge label k′. The loss function Lₜₒₜₐₗ is represented by a sum of the first loss function L_{Y}, the second loss function L_{K}, the third loss function L_{T}, and the fourth loss function Lₒᵣₜₕ, as shown in the formula (3) below. The processing circuitry 11 trains the training parameters of the causal inference model 23 so as to minimize a loss assessed by the loss function Lₜₒₜₐₗ. The training parameters specifically refer to parameters such as a weight and a bias included in the latent variable conversion layer 231, the type classification layer 232, the latent variable conversion layer 233, the effect value calculation layer 235, the effect value calculation layer 236, and the recommendation probability conversion layer 237.

Lₜₒₜₐₗ=L_{Y}+L_{K}+L_{T}+Lₒᵣₜₕ (3)

The first loss function L_{Y} represents a regression error between the estimated effect value y(0), y(1) of each event type and the effect label y′. The second loss function L_{K} represents a crossed entropy error between the estimated recommendation probability k of each event type and the knowledge label k′. The third loss function L_{T} represents a classification error between the estimated type t and the type label t′. The fourth loss function Lₒᵣₜₕ penalizes the non-orthogonality of the latent variable ht corresponding to the estimated type t and the latent variable hy corresponding to the estimated effect value y(0), y(1).

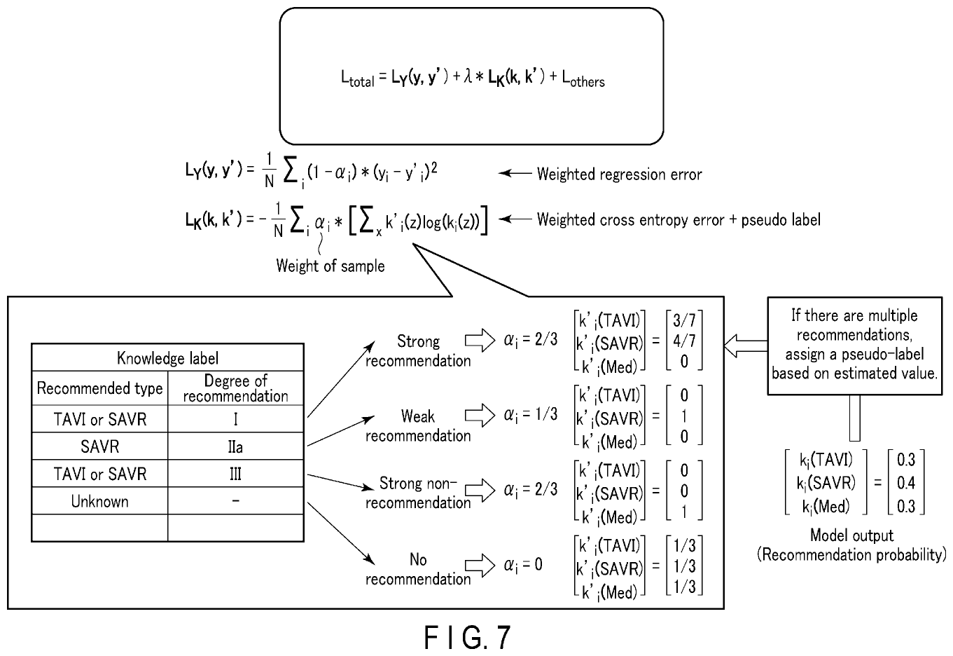

FIG. 7 schematically shows the details of the first loss function L_{Y} and the second loss function L_{K}. If focus is given to the first loss function L_{Y} and the second loss function L_{K}, the loss function L_{Total} can be expressed by the formula (4) below, as shown in the upper figure of FIG. 7. The loss function Lₒₜₕₑᵣₛ is a sum of the third loss function L_{T} and the fourth loss function Lₒᵣₜₕ in the formula (3).

Lₜₒₜₐₗ=L_{Y}(y,y′)+λ*L_{K}(k,k′)+Lₒₜₕₑᵣₛ (4)

As shown in the middle figure of FIG. 7, the first loss function L_{Y} is represented by the formula (5) below, and the second loss function L_{K} is represented by the formula (6) below. αᵢ is a weight of a training sample i. The first loss function L_{Y} is defined by averaging, using N training samples i (N representing the number of training samples), the products of a weight (1−αᵢ) and a squared difference (yᵢ−y′ᵢ)² between an estimated effect value yᵢ and an effect label y′ᵢ of the training sample i. The second loss function L_{K} is defined by averaging, using N training samples i (N representing the number of training samples i), the products of a weight αᵢ and a total of the products of a correct value of a recommendation probability (hereinafter referred to as “a correct recommendation probability”) k′ᵢ(z) of the training sample i and a natural logarithm log kᵢ(z) of an estimated recommendation probability kᵢ(z) for the event types z.

L_{Y}(y,y′)=1/NΣᵢ(1−αᵢ)*(yᵢ−y′ᵢ)² (5)

L_{K}(k,k′)=1/NΣᵢαᵢ*[Σ_{z}k′ᵢ(z)log(kᵢ(z))] (6)

The weight αᵢ has a value according to the degree of recommendation kc of the knowledge label assigned to each training sample i. In the training process, the processing circuitry 11 changes the weight (1−α₁) on the first loss function L_{Y} and the weight αᵢ on the second loss function L_{K} of each training sample i according to the degree of recommendation kc. As an example, the weight αᵢ corresponding to the degree of recommendation “I”, which means strong recommendation, has a value of “⅔”, the weight αᵢ corresponding to the degree of recommendation “IIa”, which means weak recommendation, has a value of “⅓”, the weight αᵢ corresponding to the degree of recommendation “III”, which means strong non-recommendation, has a value of “⅔”, and the weight αᵢ corresponding to the degree of recommendation “-”, which means no recommendation, has a value of “0”.

Regarding the formula (6), the correct recommendation probability k′ᵢ(z) of the event type z is determined based on a combination of the recommended type kl and the degree of recommendation kc of the knowledge label assigned to the training sample i. The correct recommendation probability k′ᵢ(z) is expressed by a vector having a combination of recommendation probabilities of predetermined event types z. The event type z is set to “TAVI”, “SAVR”, “Med”, etc. “Med” means drug treatment, which is non-invasive. If there is one recommended type z for the training sample i, the recommendation probability of this recommended type z may be set to “1”, and the recommendation probability of another recommended type z may be set to “0”.

The correct recommendation probability k′ᵢ(z) of the event type z may be determined using an estimated recommendation probability. Specifically, if multiple recommended types are selectively recommended for a single training sample i, a pseudo label based on the estimated recommendation probability kᵢ may be set as the correct recommendation probability k′ᵢ of the recommended type, as shown in FIG. 7. Let us consider, for example, the case where the estimated recommendation probability kᵢ(z) of the training sample i is represented by the formula (7) below.

For the estimated recommendation probability, the correct recommendation probability k′ᵢ(z) when the recommended type of the knowledge label is “TAVI” or “SAVR” and the degree of recommendation of the knowledge label is “I” is given by the formula (8) below, the correct recommendation probability k′ᵢ(z) when the recommended type of the knowledge label is “SAVR” and the degree of recommendation of the knowledge label is “IIa” is given by the formula (9) below, the correct recommendation probability k′ᵢ(z) when the recommended type of the knowledge label is “TAVI” or “SAVR” and the degree of recommendation of the knowledge label is “III” is given by the formula (10) below, and the correct recommendation probability k′ᵢ(z) when the recommended type of the knowledge label is “Unknown” and the degree of recommendation of the knowledge label is “-” is given by the formula (11) below.

$$[ \begin{matrix}{k}_{i} ( TAVI ) \\ {k}_{i} ( SAVR ) \\ {k}_{i} ( Med )\end{matrix} ] = [ \begin{matrix}0.3 \\ 0.4 \\ 0.3\end{matrix} ] \qquad ( 7 )$$

$$[ \begin{matrix}{k}_{i}^{′} ( TAVI ) \\ {k}_{i}^{′} ( SAVR ) \\ {k}_{i}^{′} ( Med )\end{matrix} ] = [ \begin{matrix}3 / 7 \\ 4 / 7 \\ 0\end{matrix} ] \qquad ( 8 )$$

$$[ \begin{matrix}{k}_{i}^{′} ( TAVI ) \\ {k}_{i}^{′} ( SAVR ) \\ {k}_{i}^{′} ( Med )\end{matrix} ] = [ \begin{matrix}0 \\ 1 \\ 0\end{matrix} ] \qquad ( 9 )$$

$$[ \begin{matrix}{k}_{i}^{′} ( TAVI ) \\ {k}_{i}^{′} ( SAVR ) \\ {k}_{i}^{′} ( Med )\end{matrix} ] = [ \begin{matrix}0 \\ 0 \\ 1\end{matrix} ] \qquad ( 10 )$$

$$[ \begin{matrix}{k}_{i}^{′} ( TAVI ) \\ {k}_{i}^{′} ( SAVR ) \\ {k}_{i}^{′} ( Med )\end{matrix} ] = [ \begin{matrix}1 / 3 \\ 1 / 3 \\ 1 / 3\end{matrix} ] \qquad ( 11 )$$

Regarding the formula (8), since the type “Med” is not recommended, the correct recommendation probability k′ᵢ(Med) is “0”. A value of “ 3/7” corresponding to kᵢ(TAVI) and a value of “ 4/7” corresponding to kᵢ(SAVR) shown in the formula (7) are allocated to k′ᵢ(TAVI) and k′ᵢ(SAVR), respectively. Each k′ᵢ(z) is multiplied by a weight αᵢ=“⅔” according to the degree of recommendation “I”. Regarding the formula (9), since only one recommended type, “SAVR”, is recommended, “1” is allocated to k′ᵢ(SAVR), and “0” is allocated to k′ᵢ(TAVI) and k′ᵢ(Med). Each k′ᵢ(z) is multiplied by a weight αᵢ=“1/3” according to the degree of recommendation “IIa”. Regarding the formula (10), since two types, “TAVI” and “SAVR”, are unrecommended, “0” is allocated to k′ᵢ(TAVI) and k′ᵢ(SAVR), and “1” is allocated to k′ᵢ(Med). Each k′ᵢ(z) is multiplied by a weight αᵢ=“⅔” according to the degree of recommendation “III”. Regarding the formula (11), since none of the types are recommended or unrecommended, “⅓” is evenly allocated to k′ᵢ(TAVI), k′ᵢ(SAVR), and k′ᵢ(Med). Each k′ᵢ(z) is multiplied by a weight αᵢ=“0” according to the degree of recommendation “-”.

The processing circuitry 11 trains the causal inference model 23 so as to minimize a loss assessed by the loss function Lₜₒₜₐₗ defined as described above.

Specifically, the processing circuitry 11 calculates a loss, which is a value of the loss function Lₜₒₜₐₗ, and updates, so as to reduce the calculated loss, each training parameter of the causal inference model 23 within a range of update according to an optimization method adopted. The optimization method adopted may be a stochastic gradient descent method, ADAM, or any other method. The processing circuitry 11 repeats calculation of the loss function Lₜₒₜₐₗ and updating of the training parameters until a condition for completion of updating is satisfied. Examples of the condition for completion of updating include the number of updates reaching a predetermined number of times, the accuracy of the causal inference model 23 reaching a predetermined value, and the loss reaching a value less than a threshold.

If the condition for completion of updating is satisfied, the causal inference model 23 in which the training parameters as of the satisfaction of the condition are set is output as a trained causal inference model 23. The trained causal inference model 23 may be stored in the storage device 12 or transferred to a different computer. Through the process described above, training of the causal inference model 23 is completed.

The training process described above is an example, and the embodiment is not limited thereto.

For example, the order of the acquisition of the training samples (step SA1) and the acquisition of the knowledge base (step SA2) may be reversed or simultaneous.

In the working example described above, the causal inference model is configured to output both an estimated effect value and an estimated type. However, the causal inference model only needs to output an estimated effect value and does not need to output an estimated type. In this case, the processing circuitry 11 may train the causal inference model so as to reduce a loss specified by the loss function Lₜₒₜₐₗ, which is a sum of the first loss function L_{Y} and the second loss function L_{K}. The causal inference model neither has the latent variable conversion layer 231 shown in FIG. 6 nor has the type classification layer 232 shown in FIG. 6. The training samples only need to have the feature amount, the effect label, and the knowledge label, and do not need to have the type label.

As described above, the medical information processing apparatus 1 according to the embodiment has the processing circuitry 11. The processing circuitry 11 acquires multiple training samples by implementing the sample acquisition function 111. Each of the multiple training samples includes a feature amount representing a condition of a subject, a type label of an event performed on the subject, and an effect label of the event. By implementing the knowledge base acquisition function 112, the processing circuitry 11 acquires a knowledge base independent from the multiple training samples. By implementing the assigning function 113, the processing circuitry assigns a knowledge label to at least one training sample among the multiple training samples based on the knowledge base. By implementing the training function 114, the processing circuitry 11 trains, based at least on the at least one training sample to which the knowledge label is assigned, a causal inference model that infers an effect of each type of an event. The at least one training sample to which the knowledge label is assigned includes the feature amount, the type label, the effect label, and the knowledge label.

According to the configuration described above, it is possible to generate a causal inference model that infers an effect of each type of an event by considering not only a training sample but also a knowledge base. Thus, it is possible to improve the accuracy of the causal inference model even with a small number of training samples, as compared to the case where training is performed only with a training sample.

### Application Example 1

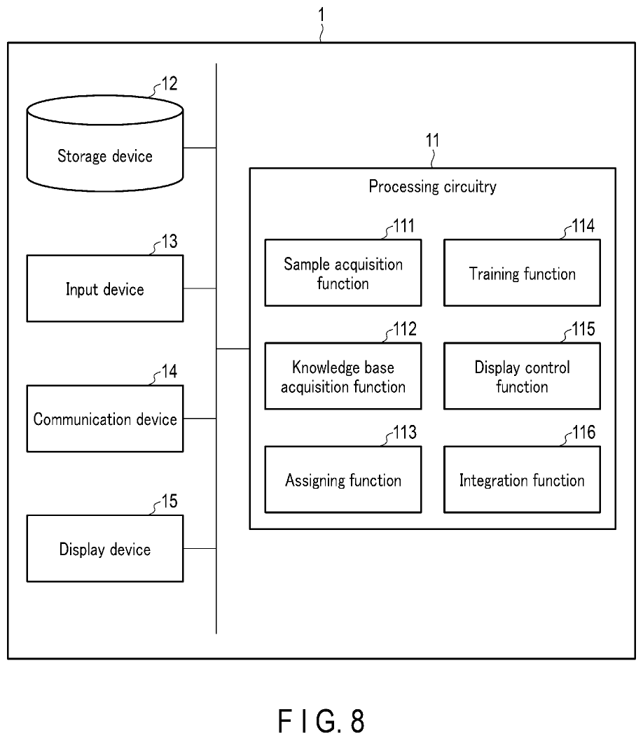

FIG. 8 shows an example of a configuration of the medical information processing apparatus 1 according to an application example 1. As shown in FIG. 8, the processing circuitry 11 according to the application example 1 implements an integration function 116 in addition to the sample acquisition function 111, the knowledge base acquisition function 112, the assigning function 113, the training function 114, and the display control function 115. By implementing the integration function 116, the processing circuitry 11 generates an integrated label that integrates the type label and the knowledge label. By implementing the training function 114, the processing circuitry 11 trains a model based on an integrated sample that includes the feature amount and the integrated label.

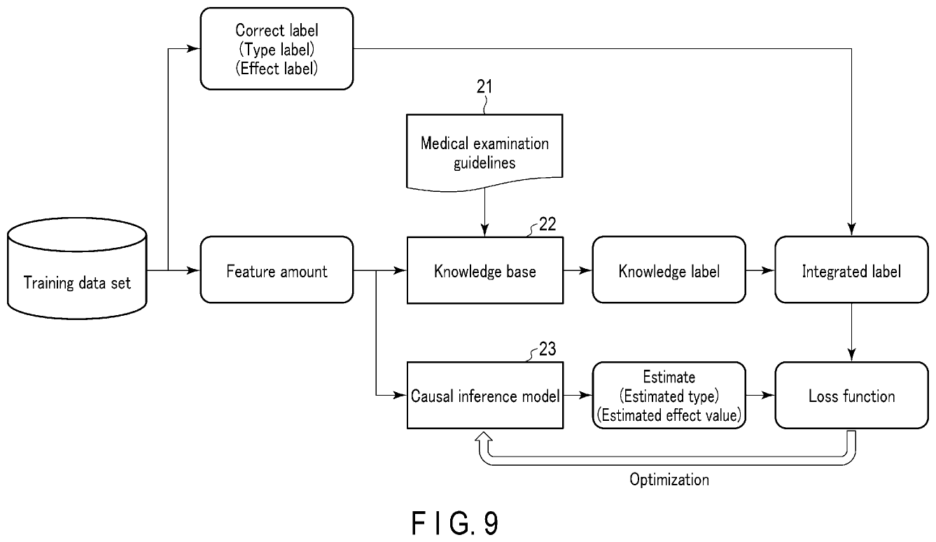

FIG. 9 schematically shows a training process according to the application example 1. In the application example 1, the processing circuitry 11 generates an integrated label by integrating a correct label and a knowledge label, and trains the causal inference model 23 through supervised training utilizing the integrated label, as shown in FIG. 9. At this time, the processing circuitry 11 trains the training parameters of the causal inference model 23 so as to reduce a loss specified by a loss function according to the application example 1. A fifth loss function according to the application example 1 is a function having the therapeutic effect value and the integrated label as variables, and is simply expressed by a mathematical formula such as L(y,c). The fifth loss function corresponds to a loss function that integrates the first loss function and the second loss function described above.

More specifically, the fifth loss function L(yᵢ, c′ᵢ) based on the therapeutic effect value yᵢ and the integrated label c′ᵢ of the training sample i is expressed by the formula (12) below.

$$L ( {y}_{i} , {c}_{i}^{′} ) = E ( {y}_{i} , {c}_{i}^{′} ) E ( {y}_{i} , {c}_{i} ) = - {\sum }_{z} {c}_{i}^{′} ( z ) log ( {y}_{i} ( z ) ) {c}_{i} ( x ) = \frac{{c}_{i}^{′} ( x )}{{\sum }_{x} {c}_{i}^{′} ( x )} {c}_{i}^{′} ( x ) = {t}_{i} ( x ) + λ * {α}_{i} * {k}_{i} ( x ) \qquad ( 12 )$$

As an example, if the correct label t′ᵢ is represented by the formula (13) below, a combination of the recommended type and the degree of recommendation of the knowledge label k′ᵢ is represented by the formula (14) below, the weight of the knowledge label is λ=0.5, and the weight of the degree of recommendation is αᵢ=1, a combination of the recommended type and the degree of recommendation of the integrated label c′ᵢ is expressed by the formula (15) below.

$$[ \begin{matrix}{t}_{i}^{′} ( TAVI ) \\ {t}_{i}^{′} ( SAVR ) \\ {t}_{i}^{′} ( Med )\end{matrix} ] = [ \begin{matrix}0 \\ 1 \\ 0\end{matrix} ] \qquad ( 13 )$$

$$[ \begin{matrix}{k}_{i}^{′} ( TAVI ) \\ {k}_{i}^{′} ( SAVR ) \\ {k}_{i}^{′} ( Med )\end{matrix} ] = [ \begin{matrix}0.5 \\ 0.5 \\ 0\end{matrix} ] \qquad ( 14 )$$

$$[ \begin{matrix}{c}_{i}^{′} ( TAVI ) \\ {c}_{i}^{′} ( SAVR ) \\ {c}_{i}^{′} ( Med )\end{matrix} ] = [ \begin{matrix}1 / 6 \\ 5 / 6 \\ 0\end{matrix} ] \qquad ( 15 )$$

As explained above, the application example 1 enables calculation of a loss function based on the integrated label. If there are multiple recommendations, a pseudo label or an unknown label may be added, as in the case of the embodiment described above.

### Application Example 2

In the embodiment described above, the training samples have a type label. This means that the training samples are existing samples. In order to improve the accuracy of the inference by the causal inference model, not only an existing training sample but also a non-existent sample, that is, a sample that does not have a type label, needs to be used. The processing circuitry according to an application example 2 generates a sample not having a type label (hereinafter referred to as an “artificial sample”), and trains the causal inference model such that the artificial sample follows the knowledge base.

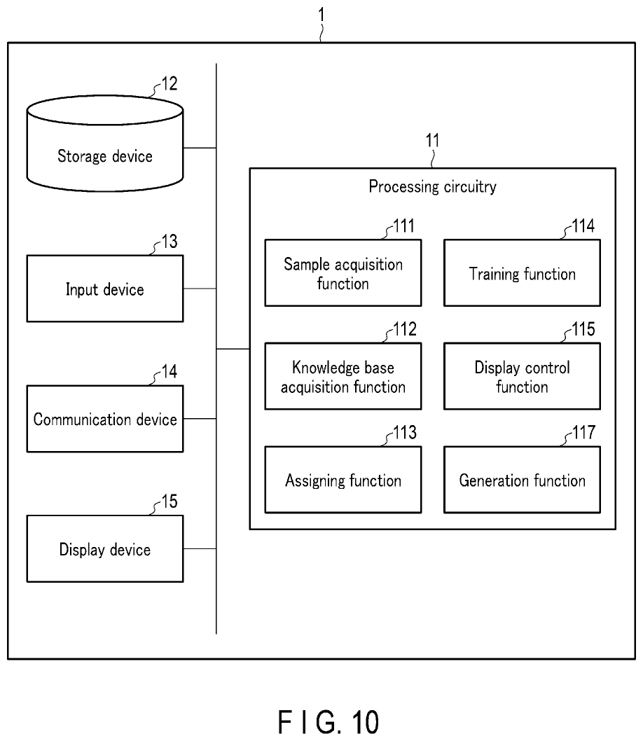

FIG. 10 shows an example of a configuration of the medical information processing apparatus 1 according to the application example 2. As shown in FIG. 10, the processing circuitry 11 according to the application example 2 implements a generation function 117 in addition to the sample acquisition function 111, the knowledge base acquisition function 112, the assigning function 113, the training function 114, and the display control function 115. The processing circuitry 11 generates an artificial sample not having a type label by implementing the generation function 117. The processing circuitry 11 assigns a knowledge label to the artificial sample by implementing the assigning function 113. Then, the processing circuitry 11 trains the causal inference model based on the training sample to which a knowledge label is assigned and the artificial sample by implementing the training function 114.

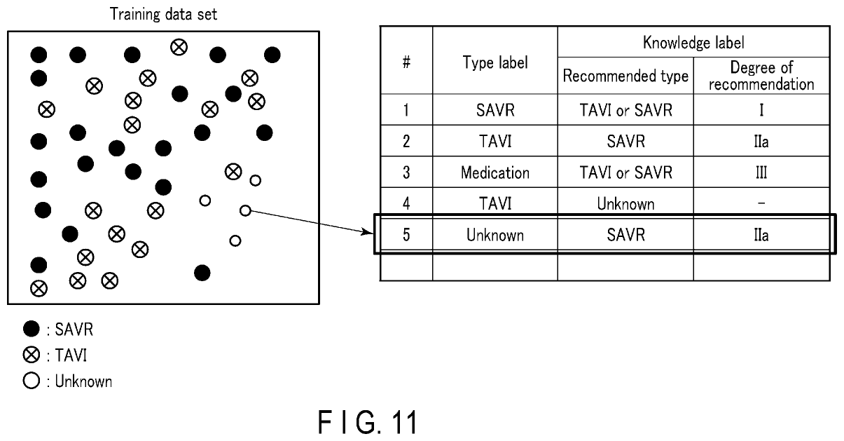

FIG. 11 schematically shows a process of generating an artificial sample. As shown in FIG. 11, there are multiple training samples in a training data set. In FIG. 11, the training sample with a type label “SAVR” is indicated by a black circle, and the training sample with a type label “TAVI” is indicated by a circled x. The artificial sample with no type label, that is, the artificial sample with a type label “Unknown”, is indicated by an open circle. To simplify the figure, the training sample with a type label “Med” is not shown. In an early stage, there is no artificial sample. A type label “Unknown” is assigned to the generated artificial sample, and a knowledge label is assigned thereto in the same manner as described in the above embodiment. In the example shown in FIG. 11, a knowledge label indicating a recommended type “SAVR” and a degree of recommendation “IIa” is assigned to the artificial sample #5.

The artificial sample is generated, for example, as follows. As an example, the processing circuitry 11 acquires, as an artificial sample, a sample generated by a facility different from a facility that generates the training samples. Then, the processing circuitry 11 determines whether or not to adopt the artificial sample based on a distance between the artificial sample and the multiple training samples in a data space.

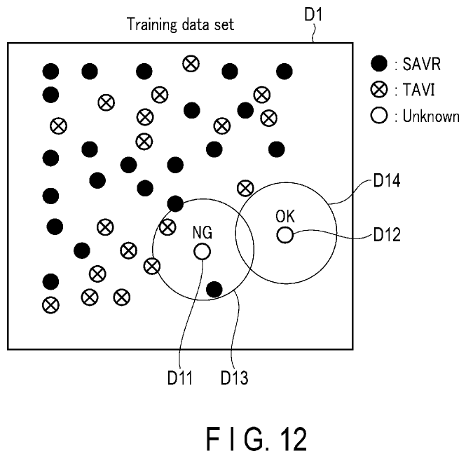

FIG. 12 schematically shows a process of determining whether or not to adopt the artificial sample. As shown in FIG. 12, there are a training sample with a type label “SAVR” and a training sample with a type label “TAVI” in a data space D1 of a training data set, as in the case shown in FIG. 11. It is assumed that an artificial sample D11 and an artificial sample D12 are generated in the data space D1. The processing circuitry 11 sets first determination spaces D13 and D14 having a predetermined first radius with the artificial samples D11 and D12 arranged in the center. If there is no training sample in the first determination spaces D13 and D14, the processing circuitry 11 determines to adopt the artificial samples D11 and D12, and if there are training samples in the first determination spaces D13 and D14, the processing circuitry 11 determines not to adopt the artificial samples D11 and D12. Specifically, with regard to the artificial sample D11, since there is a training sample in the first determination space D13, it is determined that the artificial sample D11 is not adopted (NG). With regard to the artificial sample D12, since there is no training sample in the first determination space D14, it is determined that the artificial sample D12 is adopted (OK). The first radius of the first determination spaces D13 and D14 may be set to any value in advance.

It is better not to adopt an artificial sample that is very far away from the training samples in the data space D1. In this case, the processing circuitry 11 may add a determination on whether or not to adopt an artificial sample based on a second radius longer than the first radius. Specifically, for an artificial sample determined to be adopted based on the above first radius, the processing circuitry 11 sets a second determination space having the second radius with the artificial sample arranged in the center. Then, if there is no training sample in the second determination space, the processing circuitry 11 determines to adopt the artificial sample, and if there are training samples in the second determination space, the processing circuitry 11 determines not to adopt the artificial sample. By doing so, it is possible to reject the adoption of an artificial sample that is very far away from the existing training samples. Then, it is possible to prevent degradation of the accuracy of the inference by the causal inference model.

The method of generating an artificial sample is not limited to what is described above. As an example, the processing circuitry 11 may use a randomizer to pseudo-generate an artificial sample. Specifically, a numerical value corresponding to a feature amount may be randomly generated by a randomizer. As another example, the processing circuitry 11 may use machine learning to pseudo-generate an artificial sample. A VAE (variational auto-encoder), a GAN (generative adversarial network), etc., are suitable for the machine learning. A type label “Unknown” is assigned to a generated feature amount. An artificial sample is thereby generated. In these cases as well, whether or not to adopt the artificial sample may be determined based on the first radius and/or the second radius described above.

### Application Example 3

The medical information processing apparatus according to the embodiment described above is configured to perform a process of training a causal inference model. However, the embodiment is not limited thereto. A medical information processing apparatus according to an application example 3 uses a trained causal inference model to infer a therapeutic effect value of each type of an event.

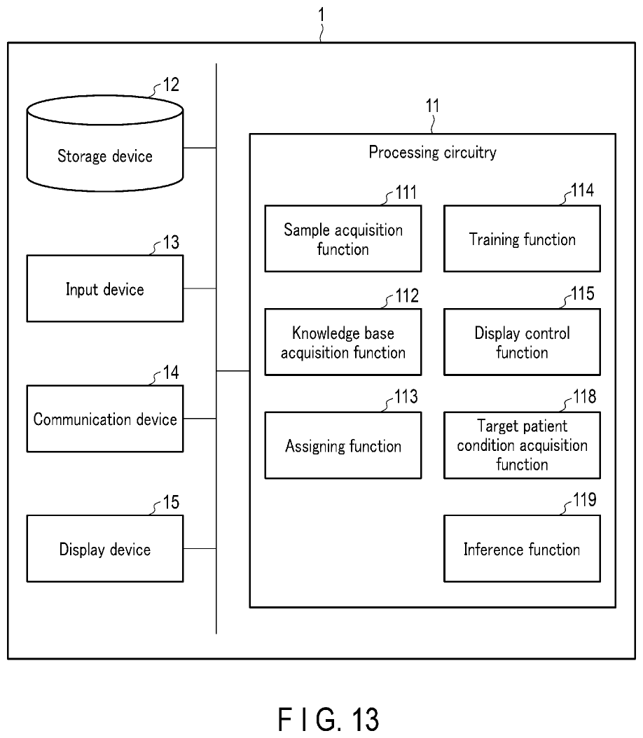

FIG. 13 shows an example of a configuration of the medical information processing apparatus 1 according to the application example 3. As shown in FIG. 13, the processing circuitry 11 according to the application example 3 implements a target patient condition acquisition function 118 and an inference function 119 in addition to the sample acquisition function 111, the knowledge base acquisition function 112, the assigning function 113, the training function 114, and the display control function 115. The processing circuitry 11 acquires a feature amount representing a condition relating to a target subject (hereinafter, this feature amount will be referred to as a “target feature amount”) by implementing the target patient condition acquisition function 118. By implementing the inference function 119, the processing circuitry 11 infers a therapeutic effect value of each event type of an event performed on a target subject based on the target feature amount and the trained causal inference model.

Hereinafter, medical information processing performed by the medical information processing apparatus 1 according to the application example 3 will be described. The medical information processing performed by the medical information processing apparatus 1 according to the application example 3 is assumed to be an inference process.

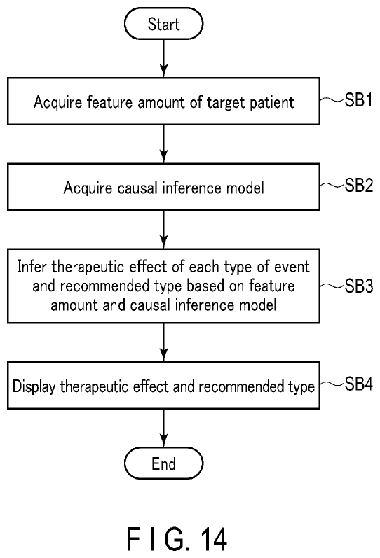

FIG. 14 shows a procedure of an inference process performed by the medical information processing apparatus 1 according to the application example 3. As shown in FIG. 14, the processing circuitry 11 first acquires a target feature amount of a target patient (step SB1). The target feature amount may be acquired from a different computer or acquired from the storage device 12.

After step SB1, the processing circuitry 11 acquires a trained causal inference model by implementing the inference function 119 (step SB2). The trained causal inference model may be acquired from a different computer or acquired from the storage device 12. The trained causal inference model is a machine-trained model trained such that a feature amount is input thereto and a therapeutic effect value and a recommended type are output therefrom.

After step SB2, by implementing the inference function 119, the processing circuitry 11 infers a therapeutic effect value of each type class and a recommended type among the type classes based on the target feature amount acquired in step SB1 and the causal inference model acquired in step SB2 (step SB3). In step SB3, the processing circuitry 11 applies the target feature amount to the causal inference model and thereby outputs a therapeutic effect value and a recommended type according to the target feature amount.

After step SB3, by implementing the display control function 115, the processing circuitry 11 causes the therapeutic effect value and the recommended type inferred in step SB3 (step SB4). In step SB4, the processing circuitry 11 causes a display screen showing the therapeutic effect value and the recommended type to be displayed on the display device 15.

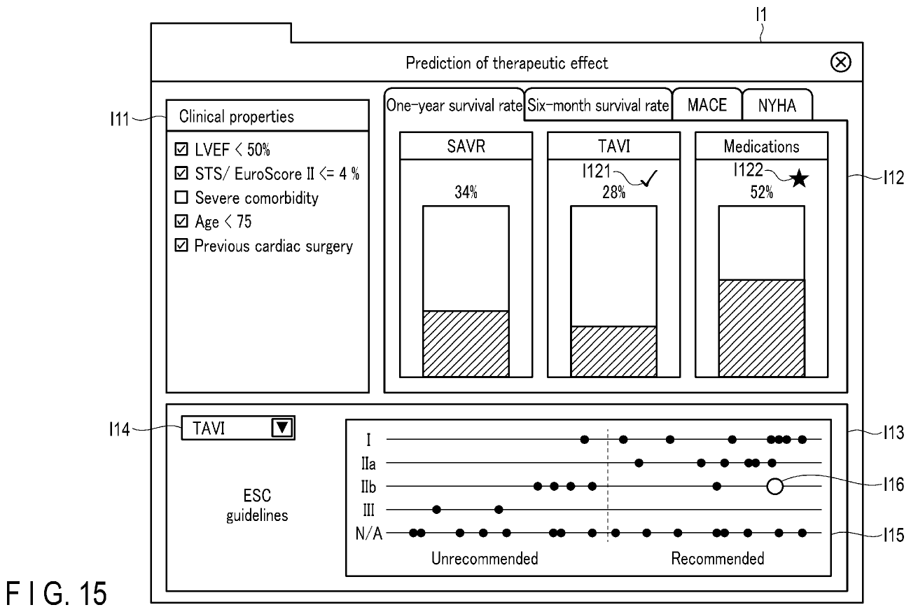

FIG. 15 shows an example of a display screen I1 of a therapeutic effect value and a recommended type. As shown in FIG. 15, the display screen I1 includes a section I11 of displaying clinical properties, a section I12 of displaying therapeutic effect values, and a section I13 of displaying results based on a knowledge base. Feature amounts that are clinical properties of a target patient are displayed in the display section I11. In FIG. 15, “LVEF”, “STS/EuroScore”, “Severe comorbidity”, “Age”, and “Previous cardiac surgery” are displayed as feature amounts.

Therapeutic effect values of respective event types are displayed in the display section I12. In FIG. 15, therapeutic effect types “one-year survival rate”, “six-month survival rate”, “MACE”, and “NYHA” are provided in the form of tabs, and therapeutic effect values are displayed for the respective event types, “SAVR”, “TAVI”, and “Medications”, in connection with each therapeutic effect type. The therapeutic effect values are shown by numerical values and bar charts. In the display section I13, a selected event type is visually highlighted. The tabs can be selected freely via the input device 13, and a user can check a therapeutic effect value of a desired therapeutic effect type by selecting a desired tab. In FIG. 15, a check mark I121 is assigned to the event type “TAVI”. Also, a star mark I122 is attached to a type that has the best therapeutic effect value among the even types.

A section I14 of selecting a type of a therapeutic effect value and a distribution chart I15 of the training samples are displayed in the display section I13. Event types that can be selected are displayed in a pull-down menu in the selection section I14, and an event type that a user is interested in is selected. In FIG. 15, the event type “TAVI” is selected. On the distribution chart I15, the probability of recommendation or non-recommendation of each training sample relating to the event type selected in the selection section I14 is displayed for each degree of recommendation. The degree of recommendation means the degree of recommendation of a recommended type assigned as a knowledge label to each training sample (i.e., the event type selected in the selection section I14). The probability of recommendation or non-recommendation means a probability of recommending the recommended type or a probability of not recommending the recommended type. The training samples are displayed using circle marks. A training sample I16 of a target patient is displayed in such a manner as to be visually distinguished from other training samples based on a color, a size, etc. The distribution chart I15 makes it possible to clearly understand the positional relationship between the training sample I16 of a target patient and training samples of other patients.

On the distribution chart I15, a user can select any training sample via the input device 13, etc. If a training sample is selected, the display section I11 and the display section I12 are updated to the display relating to the selected training sample.

The inference process according to the application example 3 is thereby completed.

The inference process described above is an example, and the embodiment is not limited thereto. For example, the above working example uses a causal inference model that outputs a therapeutic effect value and a recommended type. However, the embodiment is not limited thereto. For example, a causal inference model that outputs a therapeutic effect value may be used. This modification will be briefly explained.

The processing circuitry 11 acquires a feature amount of a target patient by implementing the target patient condition acquisition function 118. Next, with the inference function 119, the processing circuitry 11 infers multiple therapeutic effect values respectively corresponding to multiple therapeutic effect types by applying the feature amount to a causal inference model. The processing circuitry 11 then specifies a recommended type based on the multiple therapeutic effect values respectively corresponding to multiple therapeutic effect types. Thus, a recommended type can be inferred in the latter stage of the causal inference model.

In the above working example, no knowledge label is assigned to the target feature amount; however, the embodiment is not limited thereto. The processing circuitry 11 may assign a target feature amount with a knowledge label that matches the target feature amount by implementing the assigning function 113. It suffices that the assigning process is performed in the same manner as described in the above embodiment. Improved performance in the interpretation of the results of inference can be expected by assigning a knowledge label to the target feature amount. For example, the processing circuitry 11 can cause a knowledge label to be displayed on the display device 15 together with a recommended type and a therapeutic effect value, which are the results of inference. This enables a user to interpret either the results of inference or the knowledge label by comparing the results of inference and the knowledge label.

In the above working example, to be able to perform both the training process and the inference process of the causal inference model 23, the medical information processing apparatus 1 is configured such that the processing circuitry 11 has the sample acquisition function 111, the knowledge base acquisition function 112, the assigning function 113, the training function 114, the display control function 115, the target patient condition acquisition function 118, and the inference function 119. However, if the processing circuitry 11 performs the inference process, the processing circuitry 11 only needs to have the target patient condition acquisition function 118 and the inference function 119, and does not need to have the sample acquisition function 111, the knowledge base acquisition function 112, the assigning function 113, and the training function 114 for performing the training process.

### Application Example 4

In the embodiment described above, “0” is set for the weight αᵢ if the recommended type of the knowledge label is “Unknown”, which means “not known”. However, the embodiment is not limited thereto. A numerical value exceeding “0”, such as “⅓”, etc., may be set for the weight αᵢ if the recommended type of the knowledge label is “Unknown”. At this time, an unknown label “Unknown” may be provided to a classified class relating to the recommended type of the knowledge label and an estimated recommendation probability. By performing the training process in this manner, the causal inference model can also output “Unknown” as the result of estimation.

The various working examples shown above can be freely combined as appropriate. For example, estimated effect values and/or estimated types of the training samples used in the training process may be displayed on the display screen shown in FIG. 15.

According to at least one embodiment described above, it is possible to estimate a therapeutic effect with high precision even from a small amount of sample.

The term “processor” used in the above description means, for example, a CPU, a GPU, or circuitry such as an application specific integrated circuit (ASIC, a programmable logic device (e.g., a simple programmable logic device (SPLD), a complex programmable logic device (CPLD), or a field programmable gate array (FPGA)). The processor implements a function by reading and executing a program stored in storage circuitry. Note that, instead of storing the program in the storage circuitry, a configuration may be adopted in which the program is directly embedded in the circuitry of the processor. In this case, the processor implements the function by reading and executing the program incorporated into the circuit. On the other hand, if the processor is an ASIC, for example, its functions are directly incorporated into the circuitry of the processor as logic circuitry, instead of a program being stored in the storage circuitry. Each processor of the present embodiment is not limited to being configured as single circuitry; multiple sets of independent circuitry may be integrated into a single processor that implements its functions. Besides, the structural elements in FIG. 1, FIG. 8, FIG. 10 and FIG. 13 may be integrated into one processor, and the processor may implement the functions of the structural elements.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to:
acquire multiple training samples, each of the multiple training samples including a feature amount representing a condition of a subject, a type label of an event performed on the subject, and an effect label of the event;
acquire a knowledge base independent from the multiple training samples;
assign a knowledge label to at least one training sample among the multiple training samples based on the knowledge base; and
train, based at least on the at least one training sample to which the knowledge label is assigned, a model that infers an effect of each type of an event, the at least one training sample to which the knowledge label is assigned including the feature amount, the type label, the effect label, and the knowledge label.

2. The medical information processing apparatus according to claim 1, wherein the processing circuitry is configured to train the model through multi-task training comprising estimation of the knowledge label and estimation of the effect value.

3. The medical information processing apparatus according to claim 1, wherein the knowledge label includes a recommended type of the event and a degree of recommendation.

4. The medical information processing apparatus according to claim 3, wherein
the processing circuitry is configured to train the model so as to reduce a loss assessed by a loss function, and
the loss function includes a first loss function and a second loss function, wherein the first loss function represents a regression error between an estimated effect value of each type of the event and the effect label, and the second loss function represents a crossed entropy error between an estimated recommendation probability of each type of the event and the knowledge label.

5. The medical information processing apparatus according to claim 4, wherein the processing circuitry is configured to convert the estimated effect value of each type of the event to the estimated recommendation probability.

6. The medical information processing apparatus according to claim 4, wherein the processing circuitry is configured to change a first weight on the first loss function and a second weight on the second loss function of each of the training samples according to the degree of recommendation included in the knowledge label.

7. The medical information processing apparatus according to claim 4, wherein
the loss function includes:
a third loss function that represents a classification error between an estimated type of the event and the type label; and
a fourth loss function that penalizes non-orthogonality of a latent variable corresponding to the estimated type and a latent variable corresponding to the estimated effect value.

8. The medical information processing apparatus according to claim 1, wherein
the processing circuitry is configured to:
generate an integrated label that integrates the type label and the knowledge label; and
train the model based on an integrated sample that includes the feature amount and the integrated label.

9. The medical information processing apparatus according to claim 1, wherein
the processing circuitry is configured to:
generate an artificial sample not having the type label;
assign the knowledge label to the artificial sample; and
train the model based on the at least one training sample to which the knowledge label is assigned and the artificial sample.

10. The medical information processing apparatus according to claim 9, wherein the processing circuitry is configured to acquire the artificial sample from an externally provided facility or pseudo-generate the artificial sample.

11. The medical information processing apparatus according to claim 9, wherein the processing circuitry is configured to determine whether or not to adopt the artificial sample based on a distance between the artificial sample and the multiple training samples in a data space.

12. The medical information processing apparatus according to claim 1, wherein
the processing circuitry is configured to:
acquire a target feature amount representing a condition relating to a target subject; and
infer an effect value of each type of an event performed on the target subject based on the target feature amount and the model.

13. The medical information processing apparatus according to claim 12, wherein the processing circuitry is configured to infer the effect value of each type of an event performed on the target subject and a recommended type of an event performed on the target subject.

14. The medical information processing apparatus according to claim 12, wherein the processing circuitry causes the effect value to be displayed on a display.

15. The medical information processing apparatus according to claim 3, wherein the recommended type includes an unknown label.

16. A medical information processing method comprising:
acquiring multiple training samples, each of the multiple training samples including a feature amount representing a condition of a subject, a type label of an event performed on the subject, and an effect label of the event ;
acquiring a knowledge base independent from the multiple training samples;
assigning a knowledge label to at least one training sample among the multiple training samples based on the knowledge base; and
training, based at least on the at least one training sample to which the knowledge label is assigned, a model that infers an effect of each type of an event, the at least one training sample to which the knowledge label is assigned including the feature amount, the type label, the effect label, and the knowledge label.

17. A medical information processing apparatus comprising processing circuitry configured to:
acquire a model trained based on multiple training samples, at least one of the multiple training samples including a feature amount representing a condition of a subject, a type label of an event performed on the subject, an effect label of the event, and a knowledge label based on a knowledge base independent from the multiple training samples;
acquire a target feature amount representing a condition relating to a target subject; and
infer an effect of each type of an event performed on the target subject based on the target feature amount and the model.
